(19) United States
(12) Patent Application Publication
(10) Pub. No.: US 2009/0325863 A1
(43) Pub. Date: Dec. 31, 2009

(54) Somatostatin analogs and IGF-I inhibition for breast cancer prevention

(76) Inventor: David L. Kleinberg, New York, NY (US)

Correspondence Address: KLAUBER & JACKSON, 411 HACKENSACK AVENUE, HACKENSACK, NJ, 07601, US

(21) Appl. No.: 12/456,244

(22) Filed: Jun. 12, 2009

Related U.S. Application Data
(60) Provisional application No. 61/131,934, filed on Jun. 13, 2008.

(51) Int. Cl. A61K 38/12; A61K 38/16; G01N 33/53

(52) U.S. Cl. 514/9; 514/12; 435/7.23

(57) ABSTRACT

The present invention relates generally to the use and application of compounds or agents, including somatostatin analogs, with effect on, affinity for, or specificity to SSTR3 and/or SSTR5 somatostatin receptors, particularly in the breast, for the treatment of breast hyperplasia, pre-neoplastic lesions and breast carcinoma and/or prevention or reduction of risk for breast cancer or treatment of breast cancer, including DCIS. The invention also relates to use of somatostatin analog SOM230 in treatment of breast hyperplasia and/or prevention or treatment of breast cancer. The invention includes assays and methods for screening and identifying breast hyperplasia with elevated SSTR3 and/or SSTR5 receptors and for chemotherapy and identifying compounds of use in the invention which are specific for, modulate via, or bind to SSTR3 and/or SSTR5 receptors.

### CROSS-REFERENCE TO RELATED APPLICATIONS

The present application is a non-provisional application claiming the priority of co-pending provisional application Ser. No. 61/131,934, filed Jun. 13, 2008, the disclosure of which is incorporated by reference herein in its entirety. Applicants claim the benefits of this application under 35 U.S.C. §119 (e).

### GOVERNMENTAL SUPPORT

The research leading to the present invention was supported, at least in part, by a grant from the Department of Defense, US Army Medical Research, Grant No. BC061512, Synergistic Idea Award. Accordingly, the Government has certain rights in the invention.

### FIELD OF THE INVENTION

The present invention relates generally to the use and application of compounds or agents, including somatostatin analogs, with effect on, affinity for, or specificity to SSTR3 and/or SSTR5 somatostatin receptors for the treatment of breast hyperplasia, pre-neoplastic lesions and/or prevention or reduction of risk for breast cancer or the treatment of breast cancer, including DCIS. The invention also relates to use of somatostatin analog SOM230 in treatment of breast hyperplasia and/or prevention or treatment of breast cancer.

### BACKGROUND OF THE INVENTION

Women with certain hyperplastic lesions of the breast are at high risk for breast cancer. There is clinical evidence that treatment with Tamoxifen can prevent the development of cancer by about 50%. Tamoxifen treatment is problematic in that it has many side effects and makes women almost completely estrogen deficient as if they were menopausal. This is particularly unpleasant and unacceptable for premenopausal women and some menopausal ones.

Antiestrogens or aromatase inhibitors have been employed as a means of preventing breast cancer in women with preneoplastic breast lesions such as atypical hyperplasia or ductal carcinoma in situ (DCIS). While effective, these approaches cause serious side effects and symptoms of the menopause which can be intolerable, and also a high incidence of osteoporosis. Ruan et al have proposed that inhibition of IGF-I activity might be able to substitute for estrogen inhibitors because IGF-I is essential for estrogen and progesterone action in the mammary gland (Ruan W et al (2005) Endocrinology 146(3):1170-1178).

Somatostatin and somatostatin-related peptides are a family of peptides that have broad spectrum biological actions and exert suppressive effects on a large variety of cells, functioning as endogenous growth inhibitors. Naturally-occurring peptides have a short half life because they are rapidly inactivated by endogenous peptidases and therefore efforts have been made to develop more stable peptides. The three more extensively tested analogs are SMS 201-995 (octreotide), BIM 23014 (lanreotide) and RC-160 (vapreotide) (Lamberts S W J et al (1991) Endocrin Rev 12:450-482). Somatostatins bind somatostatin receptor(s), with subtypes SSTR-1 to SSTR-5 identified, cloned, and functionally characterized (Patel Y C et al (1995) Life Sci 57:1249-1265; Patel Y C et al (1996) Metabolism 45 (suppl 1):31-38; Reisine T and Bell G I (1995) Endocrin Rev 16:427-442; Buscail L et al (1995) PNAS USA 92:1580-1584; Bell G I and Reisine T (1993) Trends Neurosci 16:34-38). Octreotide (Sandostatin^{R}) and vapreotide have a low affinity for SSTR-1, a high affinity for SSTR-2, and relatively low affinity for SSTR-3 and SSTR-5.

Somatostatin analogs have an established role in the management of patients with neuroendocrine tumors but only a potential role in the treatment of solid tumors, including breast cancer. In this tumor type in particular, somatostatin analogs showed a limited activity either when used alone or when given in combination with tamoxifen or bromocriptine. Moreover, none of the randomized trials that compared the therapeutic value of the combination of octreotide and tamoxifen versus tamoxifen alone showed any advantage in favor of combined treatment. Therefore, although the great majority of trials failed to show major side effects attributable to somatostatin analogs, the use of these compounds is limited to controlled trials (Boccardo, F. and Amoroso D. (2001) Chemotherapy 47:62-77).

The new somatostatin analog called SOM230 prevents mammary development in rats via two mechanisms gland (Ruan, W et al (2006) Mol Endocrinology 20(2):426-436). One of them is an inhibitory effect on growth hormone secretion from the pituitary which can cause reduction of serum IGF-I. The other is a direct inhibition of IGF-I action in the mammary gland as demonstrated by a reduction in IRS-1 phosphorylation in the mammary gland. It has been postulated that this effect of SOM230 is mediated by either somatostatin receptor subtype (SSTR) 3 or 5 and that this causes an increase in IGF binding protein 5 (IGFBP5) which in turn blocks the local action of IGF-I in the mammary gland (Ruan, W et al (2006) Mol Endocrinology 20(2):426-436).

There is clearly a need for improved modalities and compounds for prevention of progression to breast cancer in at-risk individuals and the treatment of breast cancer. The at-risk population includes individuals such as those with an immediate family history, individuals with atypical hyperplasia, DCIS, and/or individuals positive for correlated gene alleles such as BRCA gene markers. The compound tamoxifen, which is in use for breast cancer prevention, has significant side effects, blocking circulating estrogen and causing signs and symptoms consistent with menopause. An alternative treatment that would prevent formation of these hyperplastic lesions, and therefore cancer, but not require the patients to be estrogen deficient is warranted and needed. A significant clinical need would be met in the development and discovery of therapeutic modalities and compounds which target breast and hyperplasia in the absence of the detrimental side effect(s) of current therapy.

The citation of references herein shall not be construed as an admission that such is prior art to the present invention.

### SUMMARY OF THE INVENTION

In its broadest aspect, the present invention extends to the treatment of breast or mammary hyperplasia and the prevention or treatment of breast cancer in mammals, particularly in humans, using somatostatin analogs. In a particular aspect, somatostatin analogs which preferentially target SSTR3 and/or SSTR5 are effective. An exemplary such analog is SOM230.

In the present invention, a model of hyperplasia has been implemented and it has been determined that the hyperplasia could be inhibited by somatostatin analogs, particularly utilizing exemplary analog SOM230. SOM230 caused a reduction in the number and size of breast hyperplastic lesions and also caused a reduction in cell proliferation and increase in cell death.

In accordance with the present invention, methods for the treatment of breast hyperplasia and pre-neoplasia and the prevention or treatment of breast cancer and/or the reduction of risk for breast cancer by modulating IGF-1 in the mammary gland, particularly via somatostatin analogs which act via SSTR3 and/or SSTR5, are provided. In an aspect of the method, the treatment of ductal carcinoma in situ (DCIS), a common type of noninvasive breast cancer, is provided comprising modulating IGF-1 in the mammary gland, particularly via somatostatin analogs which have affinity for SSTR3 and/or SSTR5 receptors in the breast.

In a particular aspect, methods for the treatment of breast hyperplasia and the prevention or treatment of breast cancer and/or the reduction of risk for breast cancer by administering SOM230 are provided.

The invention provides a method for reducing breast or mammary hyperplasia in a mammal comprising administering to said mammal a somatostatin analog. In a particular such aspect, the somatostatin analog preferentially targets the SSTR3 receptor and/or the SSTR5 receptor. In an aspect of this method, the somatostatin analog is selected from SOM230, somatostatin 14, BIM23A779, AN-238, RC-121, cyclic somatostatin analog peptide, and somatostatin tumor inhibiting analog.

The invention provides a method for reducing breast or mammary hyperplasia in a mammal comprising administering to said mammal SOM230 and wherein apoptosis is increased and cell proliferation is decreased in the breast or mammary gland.

In a particular aspect of the methods of the invention, the mammal is a human.

The invention provides a method for prevention of breast cancer in a mammal with an increased risk of breast cancer and/or treatment of breast cancer in a mammal diagnosed with breast cancer comprising administering to said mammal a compound or agent that modulates IGF-1 in the mammary gland. In one aspect, the compound or agent that modulates IGF-1 in the mammary gland is a somatostatin analog. In a further aspect, the somatostatin analog preferentially targets the SSTR3 receptor and/or the SSTR5 receptor.

The invention provides a method for prevention of breast cancer in a mammal with an increased risk of breast cancer and/or treatment of breast cancer in a mammal diagnosed with breast cancer comprising administering to said mammal a somatostatin analog wherein the somatostatin analog is selected from SOM230, somatostatin 14, BIM23A779, AN-238, RC-121, cyclic somatostatin analog peptide, and somatostatin tumor inhibiting analog.

The invention further provides the use of a composition of a somatostatin analog that preferentially targets the SSTR3 receptor and/or the SSTR5 receptor in the breast for the reduction of breast or mammary hyperplasia or for prevention or treatment of breast cancer in a mammal with an increased risk of breast cancer or diagnosed with breast cancer. In one such aspect, the composition comprises a somatostatin analog selected from SOM230, somatostatin 14, BIM23A779, AN-238, RC-121, cyclic somatostatin analog peptide, and somatostatin tumor inhibiting analog.

The present invention includes a method for screening of potential compounds or agents effective to reduce breast or mammary hyperplasia and/or prevent or treat breast cancer in a mammal with an increased risk of breast cancer or diagnosed with breast cancer comprising determining one or more of the following: the binding of said compounds or agents to SSTR3 and/or SSTR5 receptors; the ability of said compounds or agents to modulate the activity of SSTR3 and/or SSTR5 receptors; and the ability of said compounds or agents to modulate IGF-1. In an aspect thereof, the potential compounds or agents are administered to a cellular sample to determine binding to or activity effect upon the SSTR3 and/or SSTR5 receptors, or activity effect upon IGF-1 by comparison with a control. In one such method, the cellular sample comprises breast cells, breast cancer cells, or mammary gland. In such method of screening, the compounds or agents may particularly be somatostatin analogs.

The invention includes an assay system for screening of potential drugs effective to modulate SSTR3 and/or SSTR5 activity of target mammalian cells (particularly breast or mammary cells) by reducing cell proliferation and/or potentiating apoptosis. In one instance, the test drug could be administered to a cellular sample to determine its binding to or effect upon the SSTR3 and/or SSTR5 receptors by comparison with a control.

In another such aspect or assay system somatostatin analogs or other test compounds are tested for their ability to reduce cell proliferation and/or increase apoptosis in a mammary or breast model system in vitro or in vivo.

The invention provides an assay system for screening of potential compounds or agents effective to reduce breast or mammary hyperplasia and/or prevent or treat breast cancer in a mammal with an increased risk of breast cancer or diagnosed with breast cancer comprising determining one or more of the following: the binding of said compounds or agents to SSTR3 and/or SSTR5 receptors; the ability of said compounds or agents to modulate the activity of SSTR3 and/or SSTR5 receptors; and the ability of said compounds or agents to modulate IGF-1, wherein said system comprises one or more cellular sample comprising breast cells, breast cancer cells, or mammary gland, and wherein the SSTR 3 receptor, SSTR5 receptor and/or IGF-1 are expressed. In one such aspect of the assay system, the compounds or agents are somatostatin analogs.

The diagnostic utility of the present invention extends to the assessment of levels of SSTR3 and/or SSTR5 in breast tissue, hyperplastic lesions, biopsies, etc. to determine the risk of the patient and/or the susceptibility of the patient to somatostatin therapy(ies).

The present invention includes a method for determining the risk of an individual for developing breast cancer or breast hyperplasia and/or the susceptibility of an individual at risk of breast cancer to somatostatin therapy comprising assessing of levels of SSTR3 and/or SSTR5 in breast tissue, hyperplastic lesions, or breast biopsies in the presence and absence of a somatostatin or somatostatin analog, whereby an individual at risk for developing breast cancer or breast hyperplasia has detectable SSTR3 and/or SSTR5 levels versus a control and wherein an individual is susceptible to somatostatin therapy if the levels of SSTR3 and/or SSTR5 are reduced in the presence of a somatostatin or somatostatin analog. In an aspect of such method, the somatostatin analog is selected from the group of SOM230, somatostatin 14, BIM23A779, AN-238, RC-121, cyclic somatostatin analog peptide, and somatostatin tumor inhibiting analog.

Other objects and advantages will become apparent to those skilled in the art from a review of the following description which proceeds with reference to the following illustrative drawings.

### BRIEF DESCRIPTION OF THE DRAWINGS

FIGS. 1A and 1B depicts histological sections through a mammary gland from a rat (A) treated with hGH 600 μg+E₂ and (B) treated with E₂ alone. Note the florid hyperplasia in the gland treated with 2 hormones (A) vs. the immature ducts in (B).

FIG. 2 provides a graph of hyperplasia in presence of growth hormone plus estradiol (GH+E) or growth hormone plus estradiol plus SOM230 (GH+E+SOM) in each of All, Florid, Moderate and Mild as determined by percent hyperplastic structures.

FIGS. 3A and 3B depicts in (A) a hyperplastic gland stained for Ki67. Many of the cells are stained, and in (B) that SOM230 caused a reduction in hyperplasia and a decrease in Ki67 staining.

FIGS. 4A and 4B depicts TUNEL staining for apoptosis. The hyperplastic gland in (A) has almost no areas of apoptosis but when SOM230 was added (B) the glands were smaller and there was more apoptosis as shown in the cells that stain brown.

FIG. 5 depicts the effect of the various treatments in representative photomicrographs of whole mounts of mammary glands with the different treatments. The arrowheads point to black area that are TEBs. They are larger in the glands treated with hGH+E₂ than in most of the others.

FIG. 6 depicts graph of inhibition on hGH induced TEB formation of various combinations of growth hormone and estradiol with SOM230, tamoxifen and octreotide. Legend: GE=hGH+E₂, GE+S=hGH+E₂+SOM230, GE+S+T=hGH+E₂+SOM230+Tamoxifen, GE+T=hGH+E₂+Tamoxifen, GE+O=hGH+E₂+Octreotide. a P<0.0002 compared with GE+S and GE+S+T, P<0.02 compared with GE+T and GE+O; b P<0.003 compared with GE+T and GE+O; c P<0.0007 compared with GE+T and GE+O.

FIG. 7 shows panels of immunostaining of 5 separate samples each of Atypical Ductal Hyperplasia (ADH) (FIG. 7, middle panel) and Ductal Carcinoma in situ (DCIS) (FIG. 7, right panel) for the presence or absence of SSTR subtype receptors 1-5 with antibodies. For positive controls human pancreatic islet cell tissue was used (FIG. 7, left panel).

FIG. 8 depicts the percentage of florid hyperplasia structures from histological sections in animals treated with human growth hormone and estrogen alone (G+E), or in combination with somatostatin (G+E+S), tamoxifen (G+E+T), or somatostatin and tamoxifen (G+E+S+T).

FIG. 9 depicts a representative photomicrograph of TUNEL staining of rat mammary gland samples in rats treated with hGH+E₂ (control, left panel) or hGH+E₂+SOM230 (right panel).

FIG. 10 depicts the effect of the IGF-1 receptor kinase inhibitor PQ401 and of somatostatin 14 on mammary development of CD female mice. Whole mounts at 21 and 28 days from control animals and at 28 days for animals treated with PQ41 for 7 days or Somatostatin 14 for 7 days are shown.

FIG. 11 shows histology at 100× and 200× of 28 day control animal and 28 day animal treated with PQ41 thrice weekly. Effects of PQ41 on branch formation in these animals is depicted.

FIG. 12 provides immunostaining for pYIRS-1 (phosphorylated IRS-1) in a representative E₂ treated control (upper left panel), an hGH+E₂ treated animal (upper right panel), an hGH E₂+SOM230 treated animal (lower left panel) and an hGH+E₂+tamoxifen treated animal (lower right panel).

FIG. 13 provides photomicrographs of mammary glands from normal developing rats 28 days of age and 28 day old animals treated with SOM230 from the time they were 21 days old, stained for mast cell protease.

FIG. 14 provides photomicrographs of mammary glands from normal developing rats 28 days of age and 28 day old animals treated with SOM230 from the time they were 21 days old, stained for pYIRS-1.

FIG. 15 provides photomicrographs of mammary glands from normal developing rats 28 days of age and 28 day old animals treated with SOM230 from the time they were 21 days old, immunostained for VEGF.

FIG. 16 provides photomicrographs of mammary glands from normal developing rats 28 days of age and 28 day old animals treated with SOM230 from the time they were 21 days old, stained for Factor VIII.

FIG. 17 depicts the effect of SOM230 on hyperplastic lesions from eight (8) women before and after treatment. Apoptosis, determined by TUNEL assay, and cell proliferation (determined by Ki67 analysis) is graphed for each of the eight patients.

FIG. 18 depicts the effect of SOM230 on LCIS lobular neoplasia in two (2) women before and after treatment. Apoptosis, determined by TUNEL assay, and cell proliferation (determined by Ki67 analysis) is graphed for each of the two patients.

FIG. 19 provides photomicrographs of biopsy from DCIS patient before and after SOM 230 treatment. Cells are labeled in the first set of panels by TUNEL assay for apoptosis, and in the second set of panels by Ki67 staining for cell proliferation.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The terms “somatostatin analog(s)”, “SST analogs”, “somatostatin” and any variants not specifically listed, may be used herein interchangeably, and as used throughout the present application and claims refer to proteinaceous material including single or multiple proteins or non-proteinaceous materials, and extends to those proteins having somatostatin or somatosiatin-like activities, including the ability to bind to and/or otherwise modulate one or more somatostatin receptors SSTR1-SSTR5. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “somatostatin analog(s)”, “SST analogs”, “somatostatin” are intended to include within their scope proteins specifically recited herein as well as all substantially homologous analogs and allelic variations.

Somatostatins bind somatostatin receptor(s), with subtypes SSTR-1 to SSTR-5 identified, cloned, and functionally characterized (Patel Y C et al (1995) Life Sci 57:1249-1265; Patel Y C et al (1996) Metabolism 45 (suppl 1):31-38; Reisine T and Bell G I (1995) Endocrin Rev 16:427-442; Buscail L et al (1995) PNAS USA 92:1580-1584; Bell G I and Reisine T (1993) Trends Neurosci 16:34-38). Octreotide and vapreotide have a low affinity for SSTR-1, a high affinity for SSTR-2, and moderate affinity for SSTR-3, SSTR-4 and SSTR-5.

The somatostatin analog SOM230 prevents mammary development in rats via two mechanisms (Ruan, W et al (2006) Mol Endocrinology 20(2):426-436). One of them is an inhibitory effect on growth hormone secretion from the pituitary which can cause reduction of serum IGF-I. The other is a direct inhibition of IGF-I action in the mammary gland as demonstrated by a reduction in IRS-1 phosphorylation in the mammary gland. It has been postulated that this effect of SOM230 is mediated by either somatostatin receptor subtype (SSTR) 3 or 5 and that this causes an increase in IGF binding protein 5 (IGFBP5) which in turn blocks the local action of IGF-I in the mammary gland (Ruan, W et al (2006) Mol Endocrinology 20(2):426-436). Somatostatin analog SOM 230 is the subject of a patent application of Novartis (U.S. Ser. No. 10/343,288, published as US2005/0014686; corresponding to PCT/EP01/08824, published as WO 02/01092A3; priority Aug. 1, 2000). This Application describes the compound, compositions thereof, and method of preventing or treating disorders with an etiology comprising or associated with excess GH-secretion and/or excess IGF-1.

Thus, in a particular aspect of the invention is provided methods for treatment of breast hyperplasia and/or prevention of breast cancer, including reduction in the progression of hyperplastic conditions to cancer, comprising administration of one or more somatostatin analog which has affinity for SSTR3 and/or SSTR5 somatostatin receptors. The use of one or more somatostatin analog or other compound with enhanced affinity for SSTR3 and/or SSTR5 receptors, particularly versus SSTR1 and/or SSTR4 receptors in the treatment of breast or mammary hyperplasia and in the prevention or treatment of breast cancer in individuals at risk is provided. The exemplary compound SOM230 has affinity for SSTR3 and/or SSTR5 receptors. The present application demonstrates SSTR3 and SSTR5 somatostatin subtype receptors are present at highest concentrations in atypical hyperplasias and DCIS.

Somatostatin analogs and/or other compounds which bind or otherwise associate with and activate/signal the SSTR3 and/or SSTR5 receptors are suitable for use in the invention. The action of a somatostatin analog and its ability or capability to bind to or otherwise associate with SSTR3 and/or SSTR5 somatostatin receptor(s) can be determined by the skilled artisan by recognized or herein disclosed methods. Somatostatin analogs include but are not limited to BIM23A779 (Neuroendocrinology 83:258-263, 2006), AN-238 (Clin Cancer Research 7:2854-2861, 2001) (2-pyrrolinodoxorubicin (AN-201) linked to octapeptide carrier RC-121) (Nagy A et al (1998) Proc Natl Acad Sci USA 95:1794-1799), RC-121 (D-Phe-Cys-Tyr-D-Trp-Lys-Val-Cys-THr-NH2) (Cai, R-Z et al (1986) Proc Natl Acad Sci USA 83:1896-1900), cyclic somatostatin analog peptide which selectively binds to the SRIF receptor SSTR3 (described in U.S. Pat. No. 6,579,967), Somatostatin Tumor Inhibiting Analog (Anaspec). Nikiforovich has used molecular modeling of constrained somatostatin analog peptides to probe SSTR specificity (Nikiforovich G V et al (2007) Chemical Biology and Drug Design 69(3):163-169). These studies serve as templates for design of conformationally-constrained non-peptide scaffolds that interact with specific SSTR subtypes.

One skilled in the art can readily determine or assess the suitability of other compounds for use in the invention, including by screening in a hyperplasia model (such as described herein), or by determining its binding to and/or specificity for SSTR 3 and/or SSTR5 receptors, particularly in the breast.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

Mutations can be made in the sequence of a somatostatin and/or somatostatin analog or compound of use in the invention such as to provide adequate amino acid. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at pH 6.0)
Aspartic acid, Glutamic acid

### Basic Amino Acids (Positively Charged at pH 6.0)

### Lysine, Arginine, Histidine (at pH 6.0)

Another grouping may be those amino acids with phenyl groups:

### Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:

Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues (preferably at least about 80%, and most preferably at least about 90 or 95%) are identical, or represent conservative substitutions.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The term “preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset.

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

The term “treating” or “treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

The compounds, somatostatin or somatostatin analogs of use in the invention may be prepared in pharmaceutical compositions, with a suitable carrier and at a strength effective for administration by various means to a patient experiencing an adverse medical condition associated with breast hyperplasia and/or enhance risk of breast cancer for the treatment thereof. A variety of administrative techniques may be utilized, among them parenteral techniques such as subcutaneous, intravenous and intraperitoneal injections, catheterizations and the like. Average quantities of the compounds, somatostatins, somatostatin analogs or their subunits may vary and in particular should be based upon the recommendations and prescription of a qualified physician or veterinarian.

Also, antibodies including both polyclonal and monoclonal antibodies, and drugs that modulate the production or activity of the somatostatins, and/or somatostatin receptors, particularly SSTR3 and/or SSTR5, may possess certain diagnostic applications and may for example, be utilized for the purpose of detecting and/or measuring conditions such as viral infection or the like. For example, the somatostatins, somatostatin analogs or their receptors may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of the somatostatin analogs of the invention may be discovered or synthesized, and may be used in diagnostic and/or therapeutic protocols.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced against somatostatins, and/or somatostatin receptor peptides can be screened for various properties; i.e., isotype, epitope, affinity, etc. Such monoclonals can be readily identified in activity assays. High affinity antibodies are also useful when immunoaffinity purification of native or recombinant somatostatins, somatostatin analogs or somatostatin receptors is possible or warranted.

Preferably, the anti-somatostatin or SSTR antibody used in the diagnostic methods of this invention is an affinity purified polyclonal antibody. More preferably, the antibody is a monoclonal antibody (mAb). In addition, it is preferable for the anti-somatostatin or SSTR antibody molecules used herein be in the form of Fab, Fab′, F(ab′)₂ or F(v) portions of whole antibody molecules.

Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

Methods for producing monoclonal antibodies are also well-known in the art. See Niman et al., Proc. Natl. Acad. Sci. USA, 80:4949-4953 (1983). Typically, the somatostatin, somatostatin analogs or SSTR or a peptide analog is used either alone or conjugated to an immunogenic carrier, as the immunogen in the before described procedure for producing anti-somatostatin, somatostatin analogs or SSTR monoclonal antibodies. The hybridomas are screened for the ability to produce an antibody that immunoreacts with the somatostatin, somatostatin analogs or SSTR.

The present invention further contemplates therapeutic compositions useful in practicing the therapeutic methods of this invention. A subject therapeutic composition includes, in admixture, a pharmaceutically acceptable excipient (carrier) and one or more of a somatostatin, somatostatin analogs, polypeptide analog thereof or fragment thereof, as described herein as an active ingredient.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or cell modulation desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

The presence of SSTR3 and/or SSTR5 in cells can be ascertained by the usual immunological procedures applicable to such determinations. A number of useful procedures are known. Three such procedures which are especially useful utilize either the somatostatin, somatostatin analog or SSTR labeled with a detectable label, antibody Ab₁ labeled with a detectable label, or antibody Ab₂ labeled with a detectable label. The procedures may be summarized by the following equations wherein the asterisk indicates that the particle is labeled, and “˜” stands for the somatostatin, somatostatin analog or SSTR:

˜*+Ab₁=˜*Ab₁ A.

˜+Ab*=˜Ab₁* B.

˜+Ab₁+Ab₂*=˜Ab₁Ab₂* C.

The procedures and their application are all familiar to those skilled in the art and accordingly may be utilized within the scope of the present invention. The “competitive” procedure, Procedure A, is described in U.S. Pat. Nos. 3,654,090 and 3,850,752. Procedure C, the “sandwich” procedure, is described in U.S. Pat. Nos. RE 31,006 and 4,016,043. Still other procedures are known such as the “double antibody,” or “DASP” procedure.

In each instance, the somatostatin, somatostatin analog or SSTR forms complexes with one or more antibody(ies) or binding partners and one member of the complex is labeled with a detectable label. The fact that a complex has formed and, if desired, the amount thereof, can be determined by known methods applicable to the detection of labels.

It will be seen from the above, that a characteristic property of Ab₂ is that it will react with Ab₁. This is because Ab₁ raised in one mammalian species has been used in another species as an antigen to raise the antibody Ab₂. For example, Ab₂ may be raised in goats using rabbit antibodies as antigens. Ab₂ therefore would be anti-rabbit antibody raised in goats. For purposes of this description and claims, Ab₁ will be referred to as a primary or anti-somatostatin, somatostatin analog or SSTR antibody, and Ab₂ will be referred to as a secondary or anti-Ab₁ antibody.

The labels most commonly employed for these studies are radioactive elements, enzymes, chemicals which fluoresce when exposed to ultraviolet light, and others.

The present invention relates generally to the use and application of compounds or agents, including somatostatin analogs, with effect on, affinity for, or specificity to SSTR3 and/or SSTR5 somatostatin receptors, particularly in the breast, for the treatment of breast hyperplasia, pre-neoplastic lesions and/or prevention or reduction of risk for breast cancer. The invention also relates to use of somatostatin analog SOM230 in treatment of breast hyperplasia and/or prevention or treatment of breast cancer. The invention also relates to use of somatostatin analog SOM230 in treatment of DCIS breast cancer.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

The somatostatin receptor(s) or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

A particular assay system developed and utilized in accordance with the present invention, is known as a receptor assay. In a receptor assay, the material to be assayed is appropriately labeled and then certain cellular test colonies are inoculated with a quantity of both the labeled and unlabeled material after which binding studies are conducted to determine the extent to which the labeled material binds to the cell receptors. In this way, differences in affinity between materials can be ascertained.

Accordingly, a purified quantity of the somatostatin, somatostatin analog or SSTR may be radiolabeled and combined, for example, with antibodies or other inhibitors thereto, after which binding studies would be carried out. Solutions would then be prepared that contain various quantities of labeled and unlabeled uncombined somatostatin, somatostatin analog or SSTR, and cell samples would then be inoculated and thereafter incubated. The resulting cell monolayers are then washed, solubilized and then counted in a gamma counter for a length of time sufficient to yield a standard error of <5%. These data are then subjected to Scatchard analysis after which observations and conclusions regarding material activity can be drawn.

While the foregoing is exemplary, it illustrates the manner in which a receptor assay may be performed and utilized, in the instance where the cellular binding ability of the assayed material may serve as a distinguishing characteristic.

An assay useful and contemplated in accordance with the present invention is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluminescence is then measured photometrically, and dose response curves are obtained and compared to those of known ligands. The foregoing protocol is described in detail in U.S. Pat. No. 4,981,784 and PCT International Publication No. WO 88/03168, for which purpose the artisan is referred.

In a further embodiment of this invention, commercial test kits suitable for use by a medical specialist may be prepared to determine the presence or absence of predetermined somatostatin, somatostatin analog or SSTR activity or predetermined somatostatin, somatostatin analog or SSTR activity capability in suspected target cells. In accordance with the testing techniques discussed above, one class of such kits will contain at least the labeled somatostatin, somatostatin analog or SSTR or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence or capability of cells for predetermined somatostatin, somatostatin analog or SSTR activity, comprising:

(a) a predetermined amount of at least one labeled immunochemically reactive component obtained by the direct or indirect attachment of the somatostatin, somatostatin analog or SSTR or a specific binding partner thereto, to a detectable label;

(b) other reagents; and

(c) directions for use of said kit.

More specifically, the diagnostic test kit may comprise:

(a) a known amount of the somatostatin, somatostatin analog or SSTR as described above (or a binding partner) generally bound to a solid phase to form an immunosorbent, or in the alternative, bound to a suitable tag, or plural such end products, etc. (or their binding partners) one of each;

(b) if necessary, other reagents; and

(c) directions for use of said test kit.

In a further variation, the test kit may be prepared and used for the purposes stated above, which operates according to a predetermined protocol (e.g. “competitive,” “sandwich,” “double antibody,” etc.), and comprises:

(a) a labeled component which has been obtained by coupling the somatostatin, somatostatin analog or SSTR to a detectable label;

(b) one or more additional immunochemical reagents of which at least one reagent is a ligand or an immobilized ligand, which ligand is selected from the group consisting of:
(i) a ligand capable of binding with the labeled component (a);
(ii) a ligand capable of binding with a binding partner of the labeled component (a);
(iii) a ligand capable of binding with at least one of the component(s) to be determined; and
(iv) a ligand capable of binding with at least one of the binding partners of at least one of the component(s) to be determined; and

(c) directions for the performance of a protocol for the detection and/or determination of one or more components of an immunochemical reaction between the somatostatin, somatostatin analog or SSTR and a specific binding partner thereto.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

We previously noted that a somatostatin analog called SOM230 inhibited insulin-like growth factor-1 action in mammary gland development (Ruan, W et al (2006) Mol Endocrinology 20(2):426-436). Not only did it work by its known activity by inhibiting growth hormone production from the pituitary gland but it was also found to have an independent IGF-I inhibitory effect on the mammary gland itself, via a pituitary independent mechanism. The reason this is important is that IGF-I is essential in order for estrogen and progesterone to work on the mammary gland (Ruan W et al (1995) Endocrinology 146(3):1170-1178). Therefore we reasoned that if we could show that IGF-I inhibition is as or more effective than antiestrogens (eg Tamoxifen) in preventing hyperplasia and eventual breast cancer development, this would provide a valid and improved approach to breast cancer prevention and treatment.

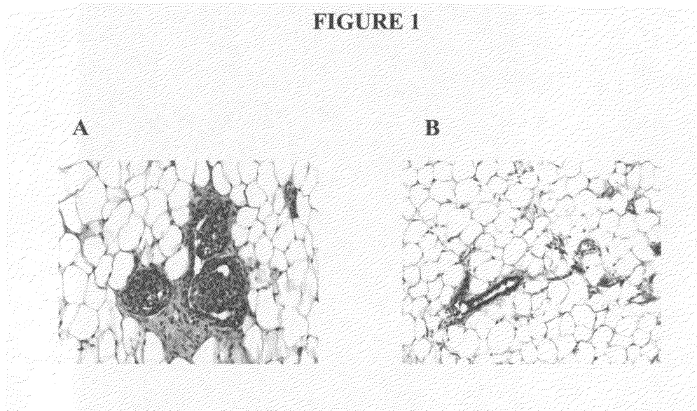

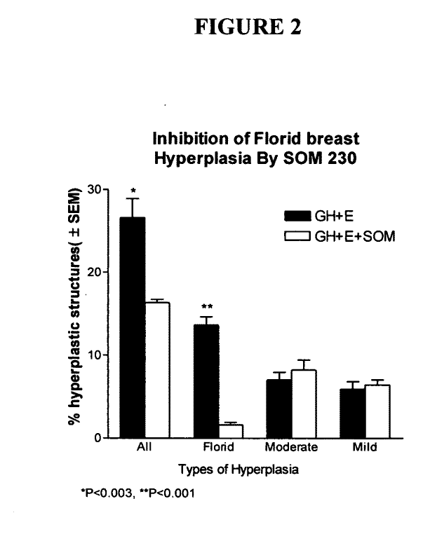

To test whether SOM230, a drug that inhibits IGF-I action on the breast, was effective, we implemented a model of hyperplasia and then determined whether the hyperplasia could be inhibited by SOM230. We administered human growth hormone (600 mcg in alzet pumps) for a week, and gave this together with estradiol (an estrogen). At the end of 7 days florid hyperplasia developed. Mammary hyperplasia was modeled by treating hypophysectomized, oophorectomized female rats with high doses human growth hormone (hGH) (600 μg in Alzet pumps) and follicular phase concentrations of estradiol (E₂) administered in silastic capsules for 7 days. In the absence of hGH, these animals would not undergo mammary development. In animals receiving E₂ alone, there was virtually no hyperplasia. When hGH was added to E₂ there were more glands (42.2±1.4 vs. 71.9±5.4, E₂ vs. E₂+hGH, respectively). There was no florid hyperplasia with E₂ treatment. When hGH was given also, 26.6% of the glands were hyperplastic, with 50% of those being floridly hyperplastic, 7% moderate hyperplasia and 5.9% mild hyperplasia. When SOM230 was added to E₂ the total number of glands (46.0±2.8) was not significantly different from the E₂ control. Florid hyperplasia was seen in a mean of 0.8±0.2 glands, moderate hyperplasia in a mean of 3.8±0.4 and mild hyperplasia in 3.0±0.4. This is shown in histological sections (FIG. 1), graphically (FIG. 2) and in tabular form below (TABLE 1). Thus, when the Human growth hormone and estradiol combination was given together with SOM230, hyperplasia was significantly inhibited. The SOM230 caused a reduction in the number and size of breast hyperplastic lesions. It also caused a reduction in cell proliferation and an increase in cell death.

TABLE 1
Effect of SOM230 on Mammary Development and Hyperplasia
Induced By hGH and E₂

| | Total# | DH# | Florid# | Moderate# | Mild# |
|---|---|---|---|---|---|
| GH + E₂ | 71.9 ± 5.4^{a} | 18.9 ± 1.3 | 9.6 ± 0.1^{c} | 5.0 ± 0.7 | 4.2 ± 0.6 |
| GH + E₂ + SOM | 46.0 ± 2.8 | 7.5 ± 0.5^{b} | 0.8 ± 0.2 | 3.8 ± 0.4 | 3.0 ± 0.4 |
| E₂ | 42.2 ± 1.4 | 1.9 ± 0.2 | 0 | 0 | 1.9 ± 0.2^{d} |

^{a}P < 0.01 compared with GH + E₂ + SOM and E₂;
^{b}P < 0.001 compared with GH + E₂ and E_{2;}
^{c}P < 0.0001 compared with GH + E₂ + SOM and E₂;
^{d}P < 0.02 compared with GH + E₂ and GH + E₂ + SOM.

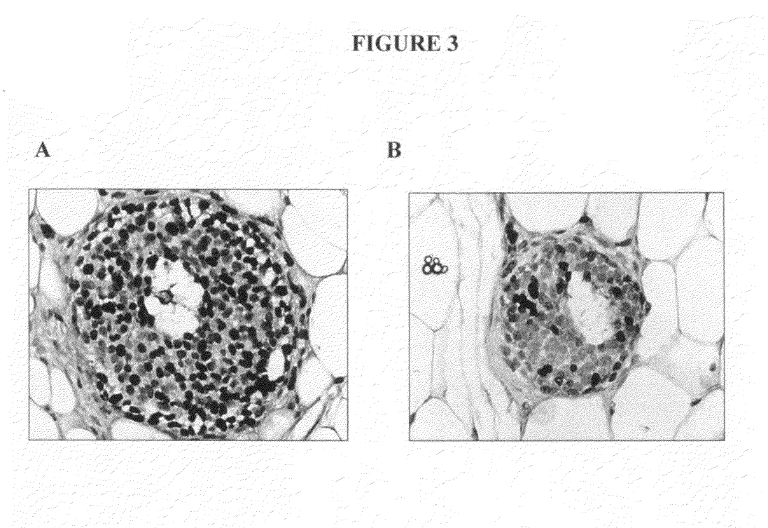

The histological sections were also analyzed for cell proliferation by Ki67 immunostaining and for apoptosis by TUNEL. Twenty five percent of cells in areas of florid hyperplasia were stained for Ki67. In areas of moderate hyperplasia 22.5% were stained, but when SOM230 was given along with the hormones this number was reduced to 14.2% (p<0.05). When the glands were normal and not hyperplastic, only 5.1% stained for Ki67. There was no significant reduction in cell proliferation in the normal tissue by SOM230. The immunostaining for Ki67 can be seen in FIG. 3.

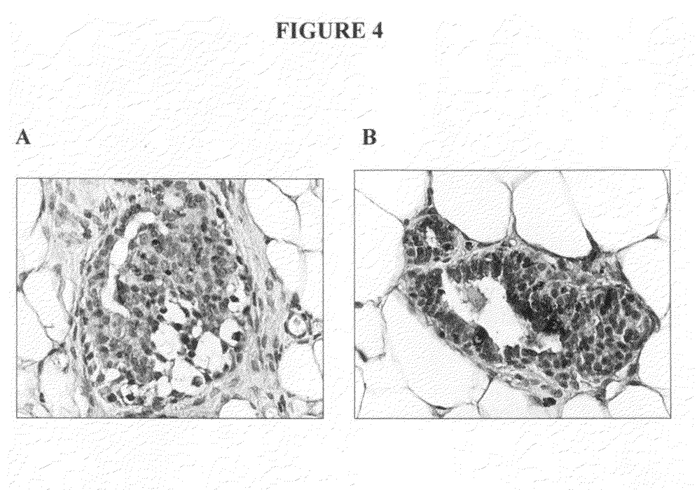

Upon staining for TUNEL, apoptosis was seen in 1.8% of floridly hyperplastic cells and 1.9% when there was moderate hyperplasia (FIG. 4). SOM230 caused an increase in apoptosis in the latter (p<0.002). SOM230 also caused an increase in apoptosis in normal gland (p<0.001).

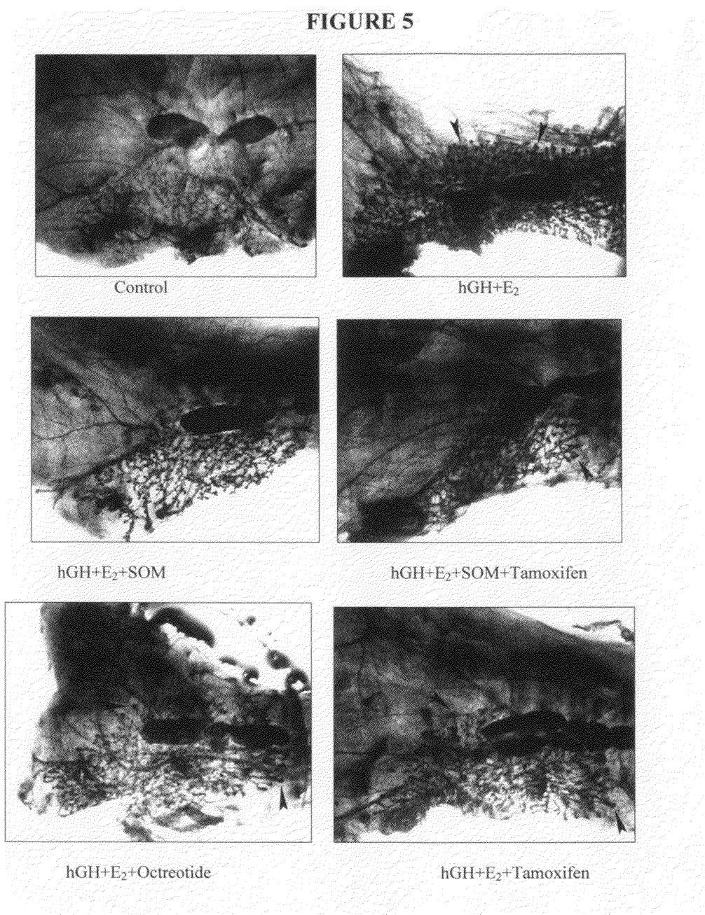

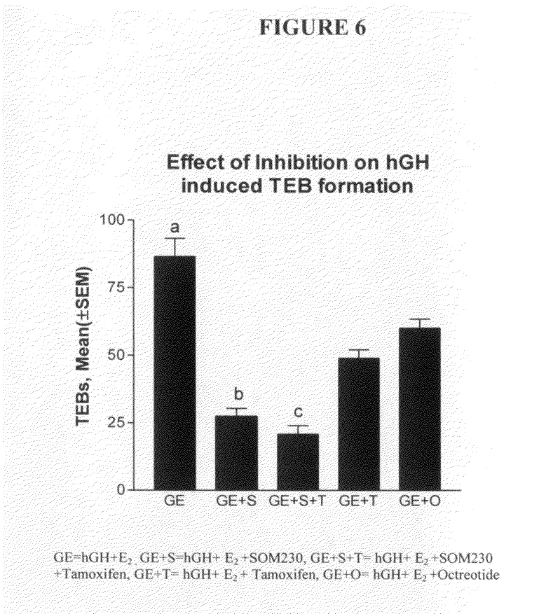

We next compared the effects of SOM230 with those of octreotide, or tamoxifen or a combination of SOM230+octreotide. The inhibitory effects of octreotide and tamoxifen on hyperplasia were significantly lower than SOM230 alone. The combination of SOM230+tamoxifen did not significantly further reduce hyperplasia over SOM230 alone. In this set of experiments the effects of SOM230 were compared to those of octreotide, tamoxifen, a combination of tamoxifen and SOM230 and a control group receiving only hGH+E₂. At the end of 7 days one lumbar mammary gland was removed for whole mount analysis and the other for histological analysis. Hyperplastic TEBs were counted in the whole mounts. Each of the therapeutic modalities had some effect on reduction of TEBs but SOM230 caused a reduction from 86.5 TEBs to 27.5 (p<0.0002), while tamoxifen reduced the number from 86 to 49. We did not do a dose response curve in this experiment but have concluded that the more profound effect of SOM230 was not significantly increased by the addition of tamoxifen. Thus we would conclude that reduction was maximal with SOM230 alone. Octreotide caused a slight but significant reduction from 86 to 60 (p<0.02). Representative photomicrographs of the whole mounts are provided in FIG. 5 and suppression by the different treatments is shown graphically in FIG. 6.

The purpose of the invention is to prevent breast cancer in women who are at high risk without making them estrogen deficient. Inhibition of IGF-I action can prevent estrogen action on the breast but does not significantly reduce circulating estrogen in women taking this drug. Therefore, this might substitute for tamoxifen or other estrogen inhibitors. This has the advantage of also preventing the action of progesterone on the mammary gland, and progesterone too may have adverse effects on causing breast cancer and hyperplasia.

### EXAMPLE 2

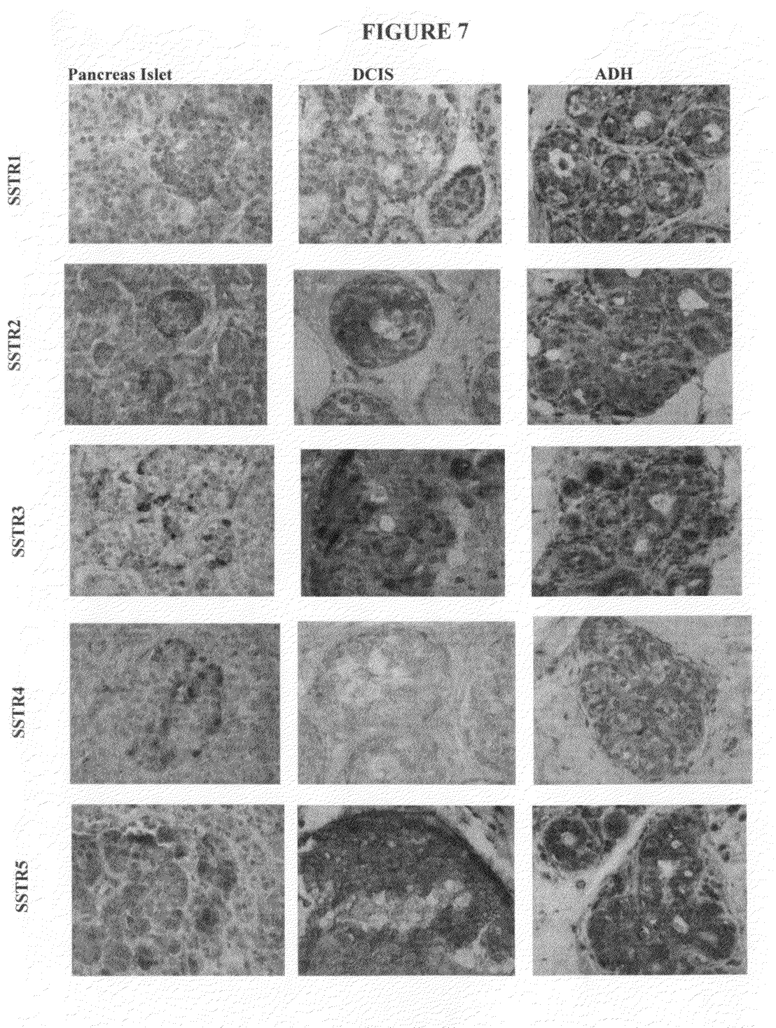

We also examined samples of normal human breast tissue and of atypical ductal hyperplasia and DCIS for presence or absence of the various somatostatin subtype receptors by immunohistochemistry. To determine whether one or more somatostatin subtype receptors was present in human breast tissue, we immuno-stained 5 separate samples each of Atypical Ductal Hyperplasia (ADH) (FIG. 7, middle panel) and Ductal Carcinoma in situ (DCIS) (FIG. 7, right panel) for the presence or absence of SSTR subtype receptors 1-5. Antibodies were generously provided by Dr. Diego Ferone, Genoa, Italy. For positive controls we used human pancreatic islet cell tissue (FIG. 7, left panel). The most intense staining in both ADH and DCIS was noted for SSTR3 and SSTR5. There was staining in cytoplasm and cell membranes.

### EXAMPLE 3

### Evidence that SOM230 can Prevent Experimental Mammary Hyperplasia by Blocking IGF-I and Thus Estrogen Action in the Mammary Gland: Preliminary Evidence for an Effect in Humans

Background: Antiestrogens or aromatase inhibitors are used for breast cancer chemoprevention. These treatments cause unpleasant side effects including hot flashes and vaginal dryness. We have previously shown that IGF-I is required to permit estrogen and progesterone action in the mammary gland. Furthermore, a novel somatostatin analog called SOM230 was found to prevent estrogen action by reducing pituitary growth hormone, but also by a direct IGF-I action inhibitory effect on the mammary gland. Menopausal symptoms would not likely to occur with SOM230 since estrogen is not lowered. Material and Methods: We have approached this issue in several ways. 1) We developed a model of hyperplasia in hypophysectomized and oophorectomized female rats. Hyperplasia was induced by treatment with 600 mcg hGH and follicular phase concentrations of estradiol. Hyperplasia was assessed histologically in sections of mammary gland. 2) To assess translatability to humans we assessed cell proliferation and apoptosis in normal human breast from reduction mammoplasties and samples of usual hyperplasia. 3) We also determined availability of SST receptors in samples of atypical ductal hyperplasia and DCIS, and 4) We have begun to determine whether SOM230 will prevent hyperplasia in humans at risk for breast cancer as it does in rats.

Results: 1) hGH and E₂ caused florid hyperplasia in rat mammary glands within a week. Twenty-six percent of gland structures exhibited hyperplasia with 50% of those showing florid hyperplasia. SOM230 reduced the number of areas of florid hyperplasia to 7.5±0.5 from 18.9±1.3 (p<0.001). This was accomplished by a reduction in cell proliferation and an increase in apoptosis. Interestingly, co-administration with tamoxifen provided no additional benefit over that of SOM230. 2) Hyperplastic lesions from humans exhibited a much higher degree of cell proliferation and a slight reduction in apoptosis compared to normal tissue. 3) We found that each somatostatin subtype receptor was present in pathology samples of atypical hyperplasias and DCIS, but SSTR3 and 5 were present in highest concentrations. We have previously shown that SSTR 3 was the receptor most likely to mediate the effect of SOM230 with SSTR5 being the next most likely. 4) In the first patient tested we found that SOM230 increased apoptosis and decreased cell proliferation in areas of lobular carcinoma in situ in an excisional biopsy as compared to an initial core biopsy.

### EXAMPLE 4

### Effect of hGH+E₂ on Formation of Hyperplastic Terminal Endbuds (TEBs): Prevention by SOM230, Tamoxifen, Octreotide And SOM230+Tamoxifen

We further assessed the effects of SOM230, tamoxifen, octreotide and a combination of tamoxifen and SOM230 on formation of TEBs in response to hGH+E₂ The effect of the different treatments on mammary gland whole mounts as judged by the number of TEBs was assessed in 6 animals treated with hGH+E₂ with and without the different treatment as above. The addition of tamoxifen to SOM230 did not provide a significant improvement in reducing TEB formation. The size of the TEBs was reduced by SOM230, as shown TABLE 2 below.

TABLE 2

| animal # | hGH + E | hGH + E + SOM |
|---|---|---|
| 1 | 48549 | 18816 |
| 2 | 38496 | 13652 |
| 3 | 42285 | 15356 |
| TEB area (μm²) × 10³ | 43.1 ± 2.9 | 15.9 ± 1.5 |
| P value | 0.002 | |

### EXAMPLE 5

### Analysis of Degree of Hyperplasia in Histological Sections

The degree of hyperplasia was analyzed in 3 histological sections at different levels in 3 test animals given GH+E₂. The different types of hyperplasia were counted. For example, the total mean number of ducts was 88.3. Of those ducts, there were 19.7 or 22.3% of total ducts that were hyperplastic. The breakdown was a mean of 10 ducts with florid hyperplasia (lumen is distended and mostly obliterated by epithelial cells), a mean of 5.3 ducts with moderate hyperplasia (an increase in cell layers to >4 cells thick), and a mean of 4.4 characterized as mild hyperplasia (no more than 4 cell thickness).

There were 4 additional treatment groups and one E₂ treated control group. The treatments were SOM230 (30 mcg/h/kg body weight), Octreotide (30 mcg/hr/kg), Tamoxifen (30 mg/21 day release pellet), and a combination of tamoxifen and SOM230. The results are shown in the TABLE 3 below.

TABLE 3
Effect of SOM230, Tamoxifen and their Combination on Mammary Development and
Hyperplasia Induced By hGH and E₂

| | Total# | DH# | Florid# | Moderate# | Mild# |
|---|---|---|---|---|---|
| GH + E₂ | 88.3 ± 6.3^{a} | 19.7 ± 1.5 | 10 ± 0.7^{d} | 5.3 ± 0.6 | 4.4 ± 0.6 |
| GH + E₂ + SOM | 61.4 ± 7.0 | 8.8 ± 0.7^{b} | 1.7 ± 0.6 | 3.8 ± 0.4 | 3.3 ± 0.3 |
| GH + E₂ + SOM + Tamo | 57.3 ± 5.4 | 8.4 ± 0.6^{c} | 1.6 ± 0.2 | 3.5 ± 0.4 | 3.4 ± 0.2 |
| GH + E₂ + Octreotide | 76.3 ± 7.3 | 15.4 ± 2.6 | 5.0 ± 0.8^{e} | 5.8 ± 1.0 | 4.6 ± 0.8 |
| GH + E₂ + Tamo | 74.2 ± 7.7 | 12.4 ± 1.5 | 4.5 ± 0.6^{e} | 4.8 ± 0.6 | 3.6 ± 0.4 |

^{a}P < 0.04 compared with GH + E₂ + SOM and GH + E₂ + SOM + Tamo;
^{b}P < 0.001 compared with GH + E₂, P < 0.05 compared with GH + E₂ + Octreotide;
^{c}P < 0.001 compared with GH + E₂, P < 0.05 compared with GH + E₂ + Octreotide and GH + E₂ + Tamo;
^{d}P < 0.0001 compared with GH + E₂ + SOM and GH + E₂ + SOM + Tamo, P < 0.001 compared with GH + E₂ + Tamo and GH + E₂ + Octreotide;
^{e}P < 0.02 compared with GH + E₂ + SOM and GH + E₂ + SOM + Tamo

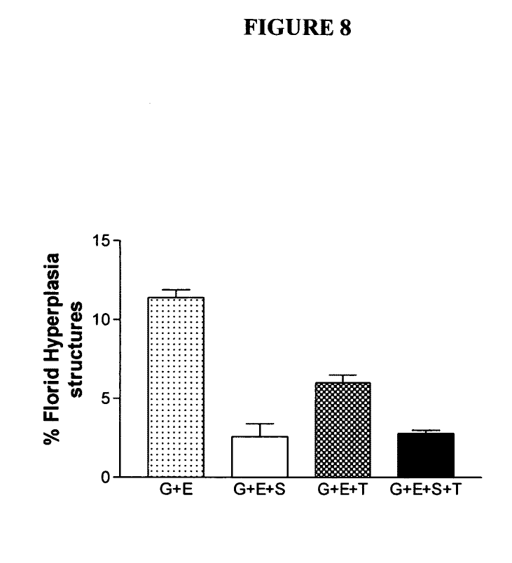

For comparison of the treatment effects, the graph of FIG. 8 shows effects of the 4 treatments on florid hyperplasia only. Note that SOM230 alone reduced the amount of florid hyperplasia from a mean of 10 to a mean of 1.7 (p<0.0001 compared to hGH+E₂). Interestingly, the addition of tamoxifen did not further reduce the hyperplasia indicating that SOM230 is at least as potent as tamoxifen and that tamoxifen does not provide additional advantage. Tamoxifen itself was significantly less effective than SOM230, although a direct dose response comparison was not done.

### EXAMPLE 6

### Effect on Cell Proliferation

We next determined the effects of the various treatment regimens on cell proliferation as assessed by Ki67 immunostaining. A remarkable decrease in cell division was measured by staining for Ki67 in the mammary glands from a hypophysectomized, oophorectomized female rat exposed to hGH+E₂+SOM 230 compared with a control treated with hGH+E₂. The percent Ki67 positive cells is depicted in TABLE 4 below.

TABLE 4
% Ki67 positive cells (3 animals)
DH structure

| | Florid | Mod + mild | Normal gland |
|---|---|---|---|
| hGH + E2 | 24.9 ± 2.2* | 22.5 ± 1.6* | 5.1 ± 0.1 |
| hGH + E2 + SOM | | 14.2 ± 2.5 | 4.3 ± 0.4 |

(*P < 0.05 compared with hGH + E2 + SOM(Mod + mild)

### EXAMPLE 7

### Effect on Apoptosis

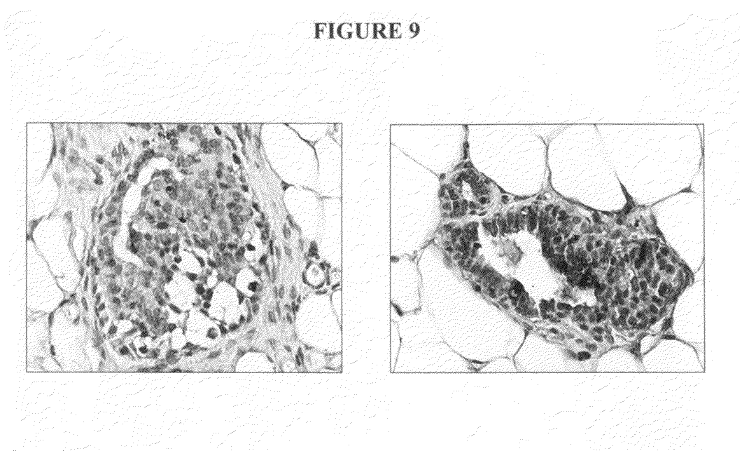

We also looked at the effect of the various treatment regimens on apoptosis. GH+E₂ inhibited apoptosis, with the results are as follows. A representative photograph of a mammary gland from a hypophysectomized, oophorectomized female rat stained for TUNEL (FIG. 9) reveals an increase in apoptosis in hGH+E₂+SOM 230 treated animals (right panel of FIG. 9) versus hGH+E₂ treatment (left panel of FIG. 9) for 7 days. The percent TUNEL labeled cells is depicted in TABLE 5 below.

TABLE 5
% TUNEL Labled cells(3 animals)
DH structure

| | Florid | Mod + mild | Normal gland |
|---|---|---|---|
| hGH + E2 | 1.8 ± 0.2* | 1.9 ± 0.2* | 2.3 ± 0.1** |
| hGH + E2 + SOM | | 5.5 ± 0.4 | 6.5 ± 0.4 |

*P < 0.002 compared with hGH + E2 + SOM(Mod + mild)
**P < 0.001 compared with hGH + E2 + SOM

### EXAMPLE 8

### Effect of PQ401 (An IGF-I Receptor Kinase Inhibitor) and Somatostatin 14 on Mammary Development

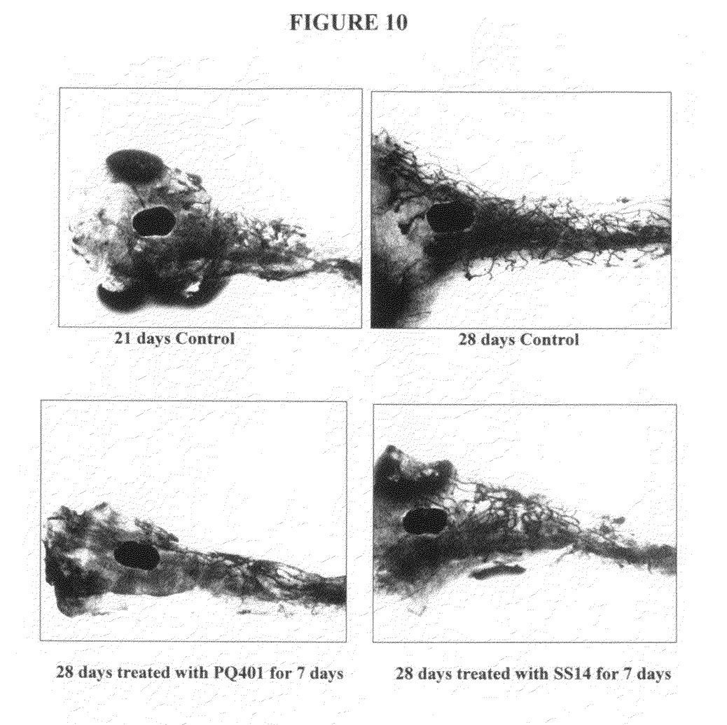

The effect of PQ401 and Somatostatin 14 (SS14) on mammary development of CD female mice was determined. FIG. 10 depicts control animals at 21 and 28 days versus 28 day old animals treated with PQ401 for 7 days and 28 day old animals treated with SS14 for 7 days. The results of TEB, branch formation, and area are depicted in TABLE 6.

TABLE 6

| | TEB | branch | area % |
|---|---|---|---|
| 21d C | 16.8 ± 2.0 | 29 ± 2.9^{d} | 23 ± 3.2^{f} |
| 28d C | 18.6 ± 1.5 | 62.6 ± 1.9 | 52.2 ± 2.0 |
| 28d PQ401 | 7.3 ± 0.9^{a} | 37 ± 7.1^{b} | 30.5 ± 5.5^{e} |
| 28d SS14 | 18.5 ± 1.5 | 41.5 ± 2.7^{c} | 36.3 ± 6.2 |

^{a}P < 0.01 compared with other three groups;
^{b}P < 0.04 compared with 28d C;
^{c}P < 0.001 compared with 28d C, P < 0.02 compared with 21d C;
^{d}P < 0.00001 compared with 28d C;
^{e}P < 0.02 compared with 28d C;
^{f}P < 0.0001 compared with 28d C.

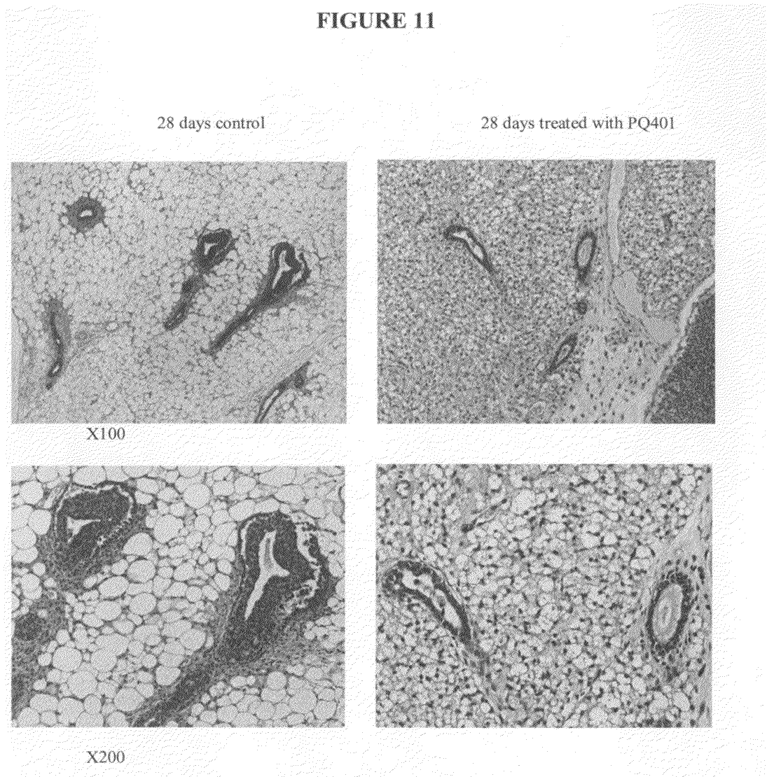

Thus, both PQ401 (100/kgbw ip thrice weekly) and SS14 (15 mcg/hr/Kg over 1 week by Alzet pump) inhibited branch formation, but PQ401 had an additional effect on inhibiting both TEB number and % gland area. This clear, effect of PQ401 was also seen histologically, as shown in FIG. 11.

### EXAMPLE 9

### Effect on Phosphorylation of IRS-1

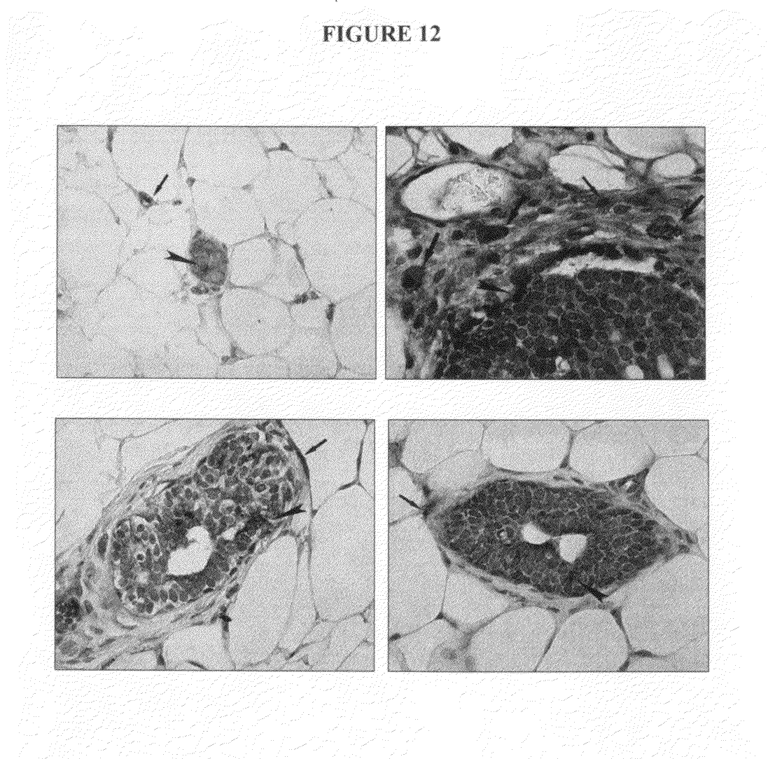

FIG. 12 depicts immunostaining for pYIRS-1 (phosphorylated IRS-1) in a representative E₂ treated control (upper left), an hGH+E₂ treated animal (upper right), an hGH+E₂+SOM230 treated animal (lower left) and an hGH+E₂+tamoxifen treated animal (lower right). The intensity of staining parallels IGF-I action as phosphorylated IRS-1 is a product of IGF-I action. In this study, staining (brown color) was most intense in response to hGH+E₂ and was inhibited by both SOM230 and tamoxifen. Note that staining in the tamoxifen treated animal is more intense than in the SOM230 treated one. This reflection of inhibition of IGF-I activity also supports the greater inhibitory effect of SOM230 over that of tamoxifen.

### EXAMPLE 10

### Mast Cells in Mammary Glands

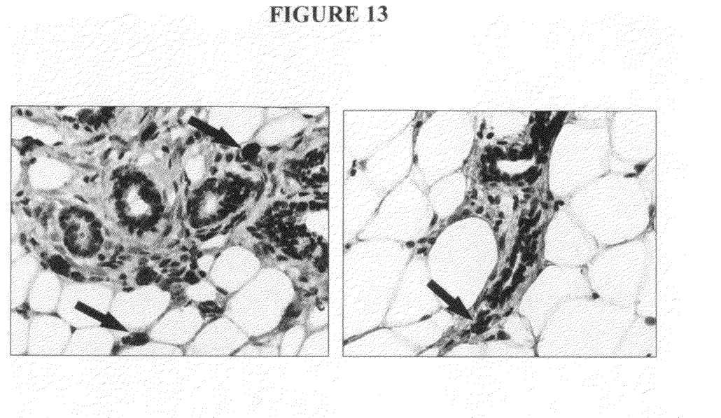

We have found for the first time that mast cells play a role in normal mammary development. Mast cells appear around developing mammary gland structures in rats. The photomicrograph of FIG. 13 shows mast cells stained by mast cell protease in normally developing rats that are 28 days of age. The same animals when they were 21 days old were given SOM230. Not only did SOM230 inhibit mammary development but it simultaneously inhibited mast cells and pYIRS-1 indicating that SOM230 inhibited these factors by inhibiting IGF-I action. The mast cells are found directly around the developing glands in the stromal compartment.

Overall, the number of mast cells/mm² was reduced by SOM230 treatment, as per TABLE 7 below. Mast cells surrounding glands and those elsewhere in the stromal compartment were both reduced.

TABLE 7

| | | Mast cell density | |
|---|---|---|---|
| | | (MC#/1 mm²) | |
| | animal# | around glands | stromal |
| normal | 3 | 11.5 ± 1.6^{a} | 3.5 ± 0.8^{c} |
| SOM treated | 3 | 3.7 ± 0.7^{b} | 1.1 ± 0.3^{d} |

^{a}P < 0.02 compared with b and c;
^{d}P < 0.05 compared with b and c.

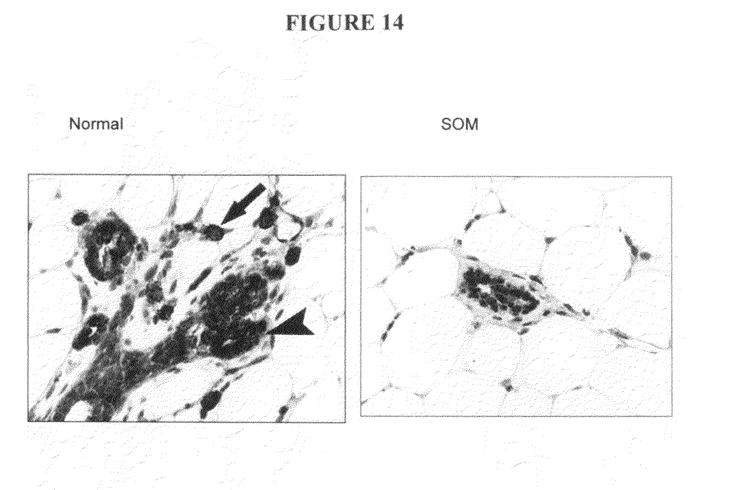

That these mast cells are directly stimulated by IGF-I is supported by the observation that phosphorylated IRS-1 was reduced in mast cells on exposure to SOM230. The action of SOM230 in mammary gland is therefore one of IGF-I action inhibition. FIG. 14 shows staining for phosphorylated IRS-1 in glands and mast cells that developed naturally in 28 day old female rats (left panel). The one on the right panel is from an animal treated with SOM230. Not only was pYIRS-I reduced in glands but mast cells were reduced as well.

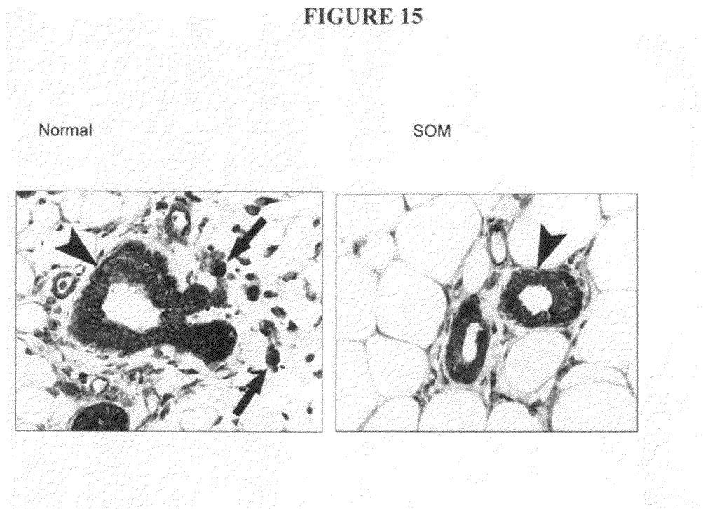

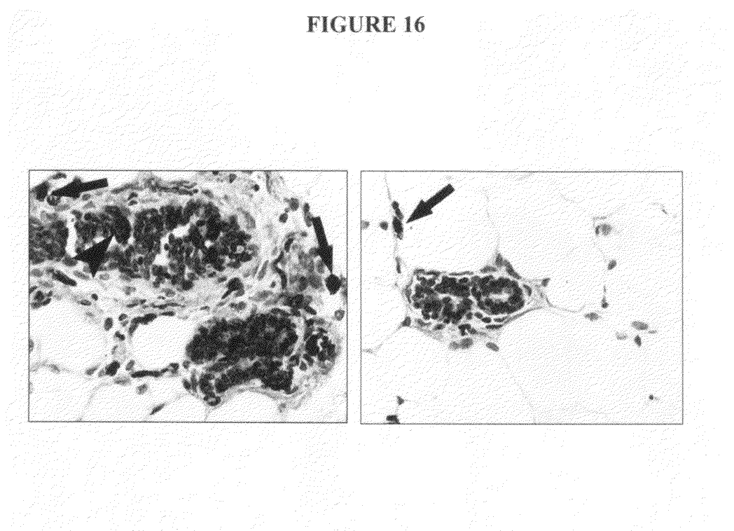

FIGS. 15 and 16 shows immunostaining for VEGF (FIG. 15) and Factor VIII (FIG. 16). In both cases SOM230 reduced expression of these factors. They were found in both gland cells and mast cells. Micro vessel density was greater in glands that were developing normally than in those in which development was inhibited by SOM230.

### EXAMPLE 11

### Effect of SOM230 in Humans

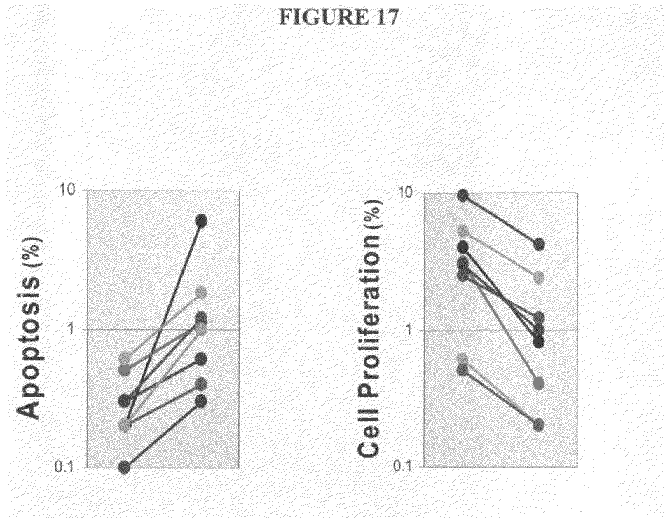

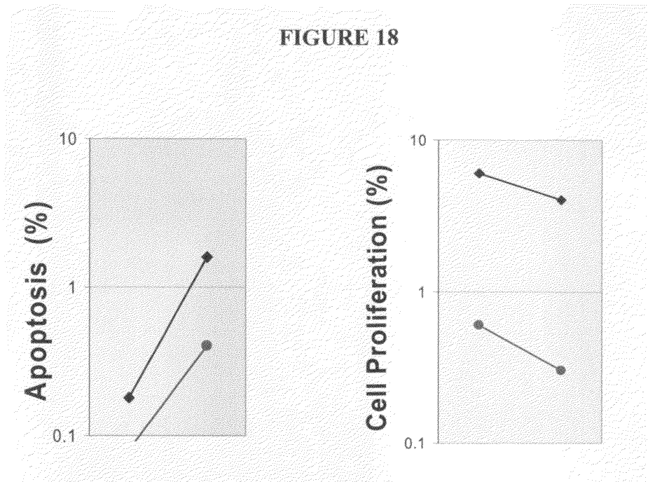

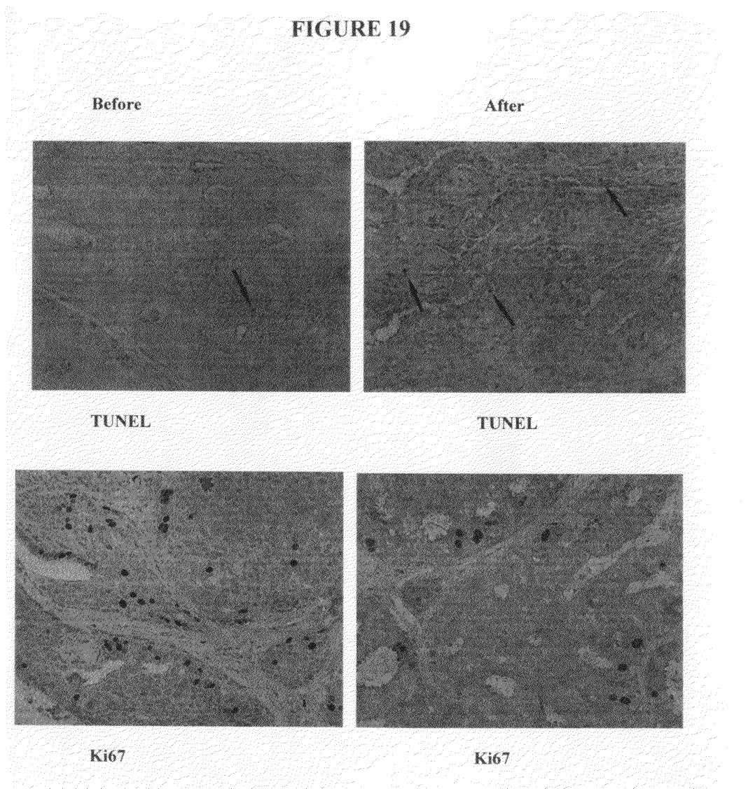

A proof of principle clinical trial is underway to further confirm whether SOM230 inhibits cell proliferation and increases apoptosis in human mammary gland as it does in rats. Data is presently available on 11 enlisted subjects. All patients had atypical hyperplasia on core biopsy and were given 600 ug SOM230 twice daily by injection. Data is available on 8 patients who had forms of hyperplasia that increase the likelihood of developing breast cancer. In each hyperplasia patient, there was inhibition of cell proliferation (assessed by Ki67 assay) and an increase in apoptosis (as assessed by TUNEL assay), based on biopsy analysis. The effect of SOM230 on apoptosis and cell proliferation in hyperplastic lesions in the 8 patients is graphed in FIG. 17. This was also true in 2 cases of LCIS, a pre-cancerous condition, and one case of ductal carcinoma in situ, a cancerous situation. Apoptosis and cell proliferation is graphed for the 2 lobular hyperplasia patients in FIG. 18. FIG. 19 provides a DCIS patient sample cell staining for TUNEL and Ki67 before and after SOM230 administration. DCIS is a very common type of noninvasive breast cancer in women. Thus, human beings respond to SOM230 by inhibiting IGF-I action in the mammary gland. Taken together with our data on relative effects of SOM230 and Tamoxifen our data provide evidence that SOM230 will be at least as effective in preventing or treating breast cancer as Tamoxifen.

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A method for reducing breast or mammary hyperplasia in a mammal comprising administering to said mammal a somatostatin analog.

2. The method of claim 1 wherein the somatostatin analog preferentially targets the SSTR3 receptor and/or the SSTR5 receptor.

3. The method of claim 1 wherein the somatostatin analog is selected from SOM230, somatostatin 14, BIM23A779, AN-238, RC-121, cyclic somatostatin analog peptide, and somatostatin tumor inhibiting analog.

4. The method of claim 1 wherein SOM230 is administered to said mammal and wherein apoptosis is increased and cell proliferation is decreased in the breast or mammary gland.

5. The method of claim 1 wherein the mammal is a human.

6. A method for prevention of breast cancer in a mammal with an increased risk of breast cancer and/or treatment of breast cancer in a mammal diagnosed with breast cancer comprising administering to said mammal a compound or agent that modulates IGF-1 in the mammary gland.

7. The method of claim 6 wherein the compound or agent that modulates IGF-1 in the mammary gland is a somatostatin analog.

8. The method of claim 7 wherein the somatostatin analog preferentially targets the SSTR3 receptor and/or the SSTR5 receptor.

9. The method of claim 7 wherein the somatostatin analog is selected from SOM230, somatostatin 14, BIM23A779, AN-238, RC-121, cyclic somatostatin analog peptide, and somatostatin tumor inhibiting analog.

10. The method of claim 7 wherein SOM230 is administered to said mammal and wherein apoptosis is increased and cell proliferation is decreased in the breast or mammary gland.

11. The method of claim 6 wherein the proliferation of DCIS breast cancer is reduced, DCIS breast cancer cells undergo apoptosis and/or DCIS is treated by the administration of a compound or agent that modulates IGF-1 in the mammary gland.

12. The method of claim 11 wherein the compound that modulates IGF-1 in the mammary gland is a somatostatin analog that preferentially targets the SSTR3 receptor and/or the SSTR5 receptor.

13. The use of a composition of a somatostatin analog that preferentially targets the SSTR3 receptor and/or the SSTR5 receptor in the breast for the reduction of breast or mammary hyperplasia or for prevention and/or treatment of breast cancer in a mammal with an increased risk of or with breast cancer.

14. The use of claim 13 wherein the composition comprises a somatostatin analog selected from SOM230, somatostatin 14, BIM23A779, AN-238, RC-121, cyclic somatostatin analog peptide, and somatostatin tumor inhibiting analog.

15. A method for screening of potential compounds or agents effective to reduce breast or mammary hyperplasia and/or prevent or treat breast cancer in a mammal with an increased risk of breast cancer comprising determining one or more of the following: the binding of said compounds or agents to SSTR3 and/or SSTR5 receptors; the ability of said compounds or agents to modulate the activity of SSTR3 and/or SSTR5 receptors; and the ability of said compounds or agents to modulate IGF-1.

16. The method of claim 15 wherein the potential compounds or agents are administered to a cellular sample to determine binding to or activity effect upon the SSTR3 and/or SSTR5 receptors, or activity effect upon IGF-1 by comparison with a control.

17. The method of claim 15 wherein the cellular sample comprises breast cells, breast cancer cells, or mammary gland.

18. The method of claim 15 wherein the compounds or agents are somatostatin analogs.

19. An assay system for screening of potential compounds or agents effective to reduce breast or mammary hyperplasia and/or prevent or treat breast cancer in a mammal with an increased risk of breast cancer comprising determining one or more of the following: the binding of said compounds or agents to SSTR3 and/or SSTR5 receptors; the ability of said compounds or agents to modulate the activity of SSTR3 and/or SSTR5 receptors; and the ability of said compounds or agents to modulate IGF-1, wherein said system comprises one or more cellular sample comprising breast cells, breast cancer cells, or mammary gland, and wherein the SSTR 3 receptor, SSTR5 receptor and/or IGF-1 are expressed.

20. The assay system of claim 19 wherein the compounds or agents are somatostatin analogs.

21. A method for determining the risk of an individual for developing breast cancer or breast hyperplasia and/or the susceptibility of an individual at risk of breast cancer to somatostatin therapy comprising assessing of levels of SSTR3 and/or SSTR5 in breast tissue, hyperplastic lesions, or breast biopsies in the presence and absence of a somatostatin or somatostatin analog, whereby an individual at risk for developing breast cancer or breast hyperplasia has elevated SSTR3 and/or SSTR5 levels versus a control and wherein an individual is susceptible to somatostatin therapy if the levels of SSTR3 and/or SSTR5 are reduced in the presence of a somatostatin or somatostatin analog.

22. The method of claim 21 wherein the somatostatin analog is selected from the group of SOM230, somatostatin 14, BIM23A779, AN-238, RC-121, cyclic somatostatin analog peptide, and somatostatin tumor inhibiting analog.
